(19) United States
(12) Patent Application Publication
(10) Pub. No.: US 2025/0057397 A1
(43) Pub. Date: Feb. 20, 2025

(54) INSTRUMENT WETTING ACCESSORY

(71) Applicant: Boston Scientific Scimed, Inc., Maple Grove, MN (US)

(72) Inventors: Juan M ALVAREZ AZPEITIA, Framingham, MA (US); Rosangel Aimee RAMOS ESPINOZA, Allston, MA (US)

(73) Assignee: Boston Scientific Scimed, Inc., Maple Grove, MN (US)

(21) Appl. No.: 18/807,687

(22) Filed: Aug. 16, 2024

Related U.S. Application Data
(60) Provisional application No. 63/520,392, filed on Aug. 18, 2023.

(51) Int. Cl. A61B 1/00; A61B 1/018

(52) U.S. Cl. CPC A61B 1/00137; A61B 1/018

(57) ABSTRACT

A biopsy cap may be operatively connected to a port of a medical device. The biopsy cap seals a working channel that is configured to allow the passing of different diameter devices. During endoscopy procedures, the tip of many devices, such as guidewires and catheters, may be wetted before use to avoid excessive dragging forces when inserting such devices through the working channel of the endoscope. A cap system comprising a cap for a port of a first medical device; and a wetting device configured to couple to a portion of the cap and wet a second medical device passing through the cap system.

### CROSS-REFERENCE TO RELATED APPLICATIONS

This application claims the benefit of priority under 35 U.S.C. § 119 from U.S. Provisional Application No. 63/520,392, filed Aug. 18, 2023, which is incorporated by reference herein in its entirety.

### TECHNICAL FIELD

Various aspects of this disclosure relate generally to medical device components, assemblies, and associated methods. In particular, aspects of this disclosure pertain devices, systems, and methods for wetting instruments at a biopsy port inlet, among other aspects.

### BACKGROUND

A biopsy cap may be operatively connected to a port of a medical device (e.g., a duodenoscope, an endoscope, a bronchoscope, an endoscopic ultrasonography (“EUS”) scope, etc.). The biopsy cap seals a working channel that is configured to allow the passing of different diameter devices. The biopsy cap also helps to prevent bioburden leakage. During endoscopy procedures, the tip of many devices, such as guidewires and catheters, may be wetted before use to avoid excessive dragging forces when inserting such devices through the working channel of the endoscope. Additionally, wetting the tip of such devices may also prevent the devices from getting caught in mucosa.

### SUMMARY

Each of the aspects disclosed herein may include one or more of the features described in connection with any of the other disclosed aspects.

In an example, a cap system may comprise a cap for a port of a first medical device; and a wetting device configured to couple to a portion of the cap and wet a second medical device passing through the cap system.

Any of the devices disclosed herein may include any of the following features, additionally or alternatively, in any combination. The wetting device may include a bottom film at a bottom of the wetting device. The bottom film may be pierced to form an opening in the bottom film. The wetting device may include a top film at a top of the wetting device. The top film may be pierced to form an opening in the top film. The cap system may further include a wetting liquid between a top film and a bottom film of the wetting device. A flange of the wetting device may define a bottom opening configured to receive the portion of the biopsy cap. The flange may be a bottom portion of the wetting device and may extend below a bottom film, wherein the bottom opening may be radially inward of the flange. The flange may define a first opening to fit around and accommodate a grip of the cap. The flange may define a second opening to fit around and accommodate a connector connecting a lid of the cap to a base of the cap. The wetting device may comprise a sponge configured to hold a wetting liquid. The sponge may be configured to be placed over and coupled to the portion of the cap. The sponge may define a through hole centrally located within the sponge and extending from a top of the sponge to a bottom of the sponge. The through hole may have a diameter of approximately 0.05 inches. The cap may further comprise a coating on at least one exterior side of the sponge.

In another example, a wetting device may comprise a tube having a substantially cylindrical shape defining an interior to receive and hold a liquid and permit a first medical device to pass through, wherein the tube is configured to be placed over a top of a base of a cap of a port of a second medical device, the tube including bottom locking structure to couple the tube to the base.

Any of the devices described herein may include any of the following features, additionally or alternatively, in any combination. The tube may further comprise a top opening configured to receive a lid of the cap and a top locking geometry to couple the tube to the lid.

In another example, a wetting device may comprise a sponge defining a through hole extending within the sponge from a top of the sponge to a bottom of the sponge, the though hole being open at the top of the sponge and open at the bottom of the sponge to allow a first medical device to pass through the through hole, wherein the sponge includes a flange at the bottom of the sponge, the flange defining a bottom opening configured to receive a portion of a cap of a port of a second medical device; and a coating on an exterior side of the sponge to inhibit leakage of a liquid absorbed by the sponge.

Any of the devices described herein may include any of the following features, additionally or alternatively, in any combination. The flange may include a first opening configured to fit around and accommodate a grip of the cap and a second opening configured to fit around and accommodate a connector connecting a lid of the cap to a base of the cap. The wetting device may have a substantially cylindrical shape.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIGS. 1A and 1B depict an exemplary medical device.

FIGS. 2A-2C depict a known biopsy cap that may be used with the exemplary medical device of FIGS. 1A-1B.

FIGS. 3A-3B depict a biopsy cap system of this disclosure.

FIGS. 4A-4B depict another biopsy cap system of this disclosure.

FIGS. 5A-5B depict another biopsy cap system of this disclosure.

FIG. 6 depicts another biopsy cap system.

FIG. 7 depicts another biopsy cap system of this disclosure.

### DETAILED DESCRIPTION

As discussed in the Background, the tip of many devices, such as guidewires and catheters, may be wetted before insertion into a biopsy cap of a medical device, to avoid excessive dragging forces and prevent the devices from getting caught in mucosa. Conventional approaches include pouring water into a kidney-shaped container that may be located on a tower or counter space. However, such approaches create an opportunity for water to be spilled onto the floor, which increases the risk of users slipping. Additionally, the open container of water can also quickly become contaminated if bioburden accidentally falls into the container. Moreover, such approaches could lead to a process complication by adding unnecessary steps to prepare the devices (e.g., pouring water into the kidney-shaped container, dipping the device into the water, etc.). As a result, a need exists for improved devices, systems, and methods for wetting the devices.

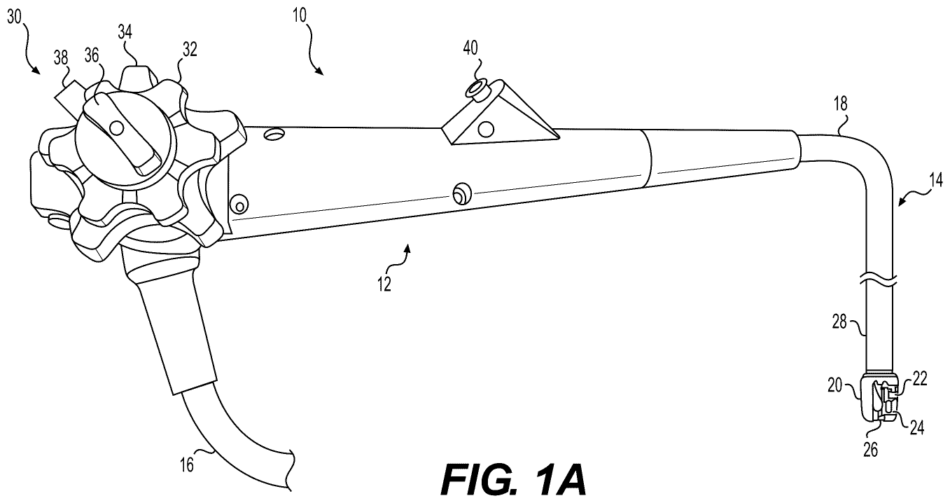

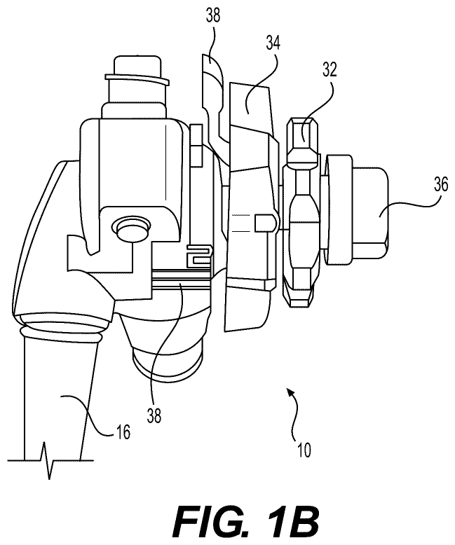

FIG. 1A depicts an exemplary duodenoscope 10 having a handle 12 and an insertion portion 14. FIG. 1B shows a proximal end of handle 12. The duodenoscope 10 may also include an umbilicus 16 for purposes of connecting the duodenoscope 10 to sources of, for example, air, water, suction, power, etc., as well as to image processing and/or viewing equipment. Although a duodenoscope may be referenced herein, it will be appreciated that the disclosure also encompasses endoscopes, bronchoscopes, gastroscopes, EUS scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

The insertion portion 14 may include a sheath or shaft 18 and a distal tip 20. The distal tip 20 may include an imaging device 22 (e.g., a camera) and a lighting source 24 (e.g., an LED or an optical fiber). The distal tip 20 may be side-facing. That is, the imaging device 22 and the lighting source 24 may face radially outward, perpendicularly, approximately perpendicularly, or otherwise transverse to a longitudinal axis of the shaft 18 and the distal tip 20. Additionally or alternatively, the distal tip 20 may include one or more imaging devices 22 that face in more than one direction. For example, a first imaging device 22 may face radially outward, and a second imaging device 22 may face distally (approximately parallel to a longitudinal axis of the distal tip 20/shaft 18).

The distal tip 20 may also include an elevator 26 for changing an orientation of a tool inserted in a working channel of the duodenoscope 10. The elevator 26 may alternatively be referred to as a swing stand, pivot stand, raising base, or any suitable other term. The elevator 26 may be pivotable via, e.g., an actuation wire or another control element that extends from the handle 12, through the shaft 14, to the elevator 26.

A distal portion of shaft 18 that is connected to the distal tip 20 may have a steerable section 28. The steerable section 28 may be, for example, an articulation joint. The shaft 18 and the steerable section 28 may include a variety of structures which are known or may become known in the art.

The handle 12 may have one or more actuators/control mechanisms 30. The control mechanisms 30 may provide control over the steerable section 28, the elevator 26, and/or may allow for provision of air, water, suction, etc. For example, the handle 12 may include the control knobs 32, 34 for left, right, up, and/or down control of a steerable section 28. For example, one of the knobs 32, 34 may provide left/right control of the steerable section 28, and the other of the knobs 32, 34 may provide up/down control of the steerable section 28. The handle 12 may further include one or more of the locking mechanisms 36 (e.g., knobs or levers) for preventing steering and/or braking of the steerable section 28 in at least one of an up, down, left, or right direction. The handle 12 may include an elevator control lever 38 (see FIG. 1B). The elevator control lever 38 may raise and/or lower the elevator 26, via connection between the lever 38 and an actuating wire that extends from the lever 38, through the shaft 18, to the elevator 26. A port 40 may allow passage of a tool through the port 40, into a working channel of the duodenoscope 10, through the sheath 18, to the distal tip 20.

In use, an operator may insert at least a portion of the shaft 18 into a body lumen of a subject. The distal tip 20 may be navigated to a procedure site in the body lumen. The operator may insert an accessory device (not shown) into the port 40, and pass the accessory device through the shaft 18 via a working channel to the distal tip 20. The accessory device may exit the working channel at the distal tip 20. The user may use the elevator control lever 38 to raise the elevator 26 and angle the accessory device toward a desired location (e.g., a papilla of the pancreatic-biliary tract). The user may use the accessory device to perform a medical procedure.

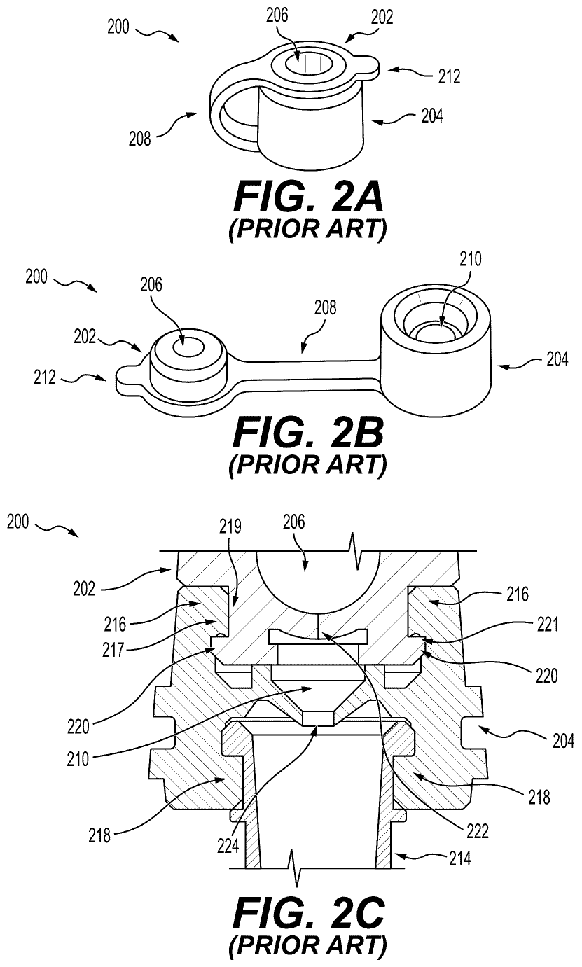

FIGS. 2A-2C depict an example of a known biopsy cap 200. FIG. 2A depicts a perspective view of the biopsy cap 200 in a closed configuration. FIG. 2B depicts a perspective view of the biopsy cap 200 in an open configuration, and FIG. 2C depicts a side, cross-sectional view of the biopsy cap 200 mounted on the access port 214. The biopsy cap 200 includes a biopsy cap lid 202 and a biopsy cap base 204 configured to attach to the access port 214. The biopsy cap 200 is an example of a cap that may be used in connection with embodiments of the disclosure, though the scope of the disclosure extends to other types and configurations of caps that mount to access ports of scopes.

The biopsy cap 200 includes a biopsy cap lid 202, a biopsy cap base 204, an upper opening 206, a biopsy cap lid connector 208, a middle opening 210, a grip 212, and a lower opening 224. The biopsy cap base 204 is configured to allow an instrument (e.g., a catheter) to pass through the biopsy cap base 204 via the middle opening 210 and into the access port 214. The biopsy cap base 204 is configured to be placed over the access port 214 via a lower base securement region 218. For example, the lower base securement region 218 includes a flange that fits into a groove of the access port 214 to secure the biopsy cap base 200 to the access port 214. The biopsy cap base 204 includes a middle opening 210 configured to receive the biopsy cap lid 202 via the upper base securement region 216. The biopsy cap lid 202 is configured to be placed into the middle opening 210 via a lid securement region 220. For example, the upper base securement region 216 has a radially-inner-facing lip 217 that is received in a radially-outward-facing groove 219 of the lid securement region 220. And a lower portion of the lid securement region 220 includes a flange 221 that is held under the lip 217 of the upper base securement region 216. Other methods of removably coupling a cap lid to a cap base are within the scope of this disclosure.

When the biopsy cap lid 202 is coupled to the biopsy cap base 204, as shown in FIG. 2C, the biopsy cap lid 202 is configured to allow an instrument (e.g., a catheter, a tool, a guidewire, etc.) to pass into the biopsy cap lid 202 via the upper opening 206, through a slit 222, into the middle opening 210, through the lower opening 224, and into the access port 214. The grip 212 is part of the biopsy cap lid 202 and extends radially outward relative to the remaining perimeter of the biopsy cap lid 202 at the top of the lid 202 in the configuration shown in FIGS. 2A and 2C. The grip 212 includes a flattened surface configured for a user to grasp to maneuver the biopsy cap lid 202.

The biopsy cap lid connector 208 is configured to join the biopsy cap lid 202 and the biopsy cap base 204. For example, one end of the biopsy cap lid connector 208 is connected to the biopsy cap lid 202, and the opposite end of the biopsy cap lid connector 208 is connected to the biopsy cap base 204. The lid connector 208 is flexible and bendable so that it can permit the biopsy cap 200 to transition between the closed configuration (FIGS. 2A and 2C) and the open configuration (FIG. 2B).

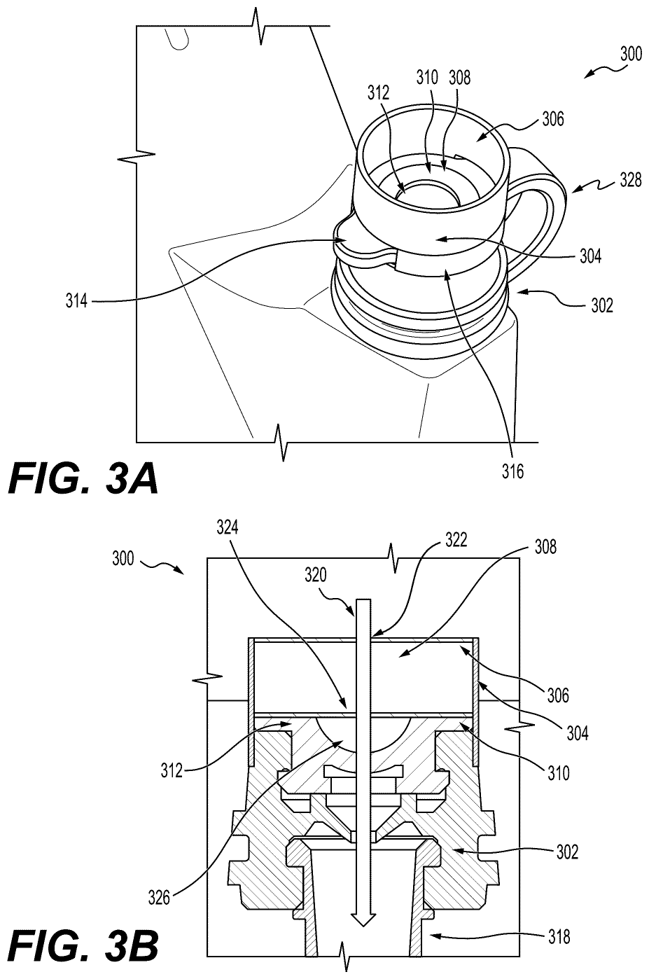

Referring to FIGS. 3A-3B, in one embodiment, a biopsy cap system 300 of this disclosure includes a biopsy cap 302 comprising the same (or similar) elements as outlined in FIGS. 2A-2C. The biopsy cap system 300 also includes a cup 304 configured to wet an instrument 320 passing through the biopsy cap system 300.

The cup 304 includes a top film 306, a wetting liquid 308, a bottom film 310, and a flange 316. The cup 304 includes a substantially cylindrical shape defining an interior to receive and hold the wetting liquid 308. The cup 304 is configured to be placed over the top 312 of the biopsy cap 302. For example, the cup 304 may include a bottom opening configured to receive the lid of the biopsy cap 302 via the flange 316. The flange 316 is a bottom portion of the cup 304 that extends past the bottom film 310 and defines the bottom opening radially inward of and within the flange 316. When fitted over the biopsy cap 302, the flange 316 is located proximal to the biopsy cap 302. The flange 316 includes two openings/slots: a first opening to fit around and accommodate the grip 314; and a second opening to fit around and accommodate the biopsy cap lid connector 328. The flange 316 has a dimension and a flexibility to friction fit to the lid of the biopsy cap 302. The flange 316 may flex slightly radially outward upon its coupling to the lid of the biopsy cap 302 to provide the friction fit. The cup 304 may be removable from the lid of the biopsy cap 312. In other embodiments, the cup 304 may be integrally molded with or otherwise manufactured as one piece with the biopsy cap 302, where the cup 304 and the biopsy cap 302 are one integral structure.

The bottom film 310 may be made out of a thin plastic and/or a silicone material so that the instrument 320 (e.g., a catheter, a guidewire, etc.) may pierce through the bottom film 310, but firm enough to hold the wetting liquid 308 prior to insertion of the instrument 320. The bottom film 310 is attached to the bottom of the cup 304, just above the flange 316, and extends across the top 312 of the biopsy cap 302 when the cup 304 is fitted to the biopsy cap 302. The bottom film 310 prevents the wetting liquid 308 from draining into the biopsy cap 302, prior to insertion of the instrument 320 into the biopsy cap system 300. Once the instrument 320 is inserted into the biopsy cap system 300 and pierces the bottom film 310, the bottom film 310 includes an opening 324 to allow an instrument 320 to pass through the bottom film 310 and into the opening 326 of the biopsy cap 302 and through the access port 318. In some embodiments, the bottom film 310 may be made out of a material and/or have a structure that allows the instrument 320 to pass through the opening 324, yet the opening 324 seals against the outside of the instrument 320 to inhibit leakage of the wetting liquid 308 into the biopsy cap 302.

The top film 306 may be made out of a thin plastic and/or a silicone material so that the instrument 320 (e.g., a catheter, a guidewire, etc.) may pierce through the top film 306, but firm enough to hold the wetting liquid 308 prior to insertion of the instrument 320. The top film 306 is attached to the top of the cup 304. The top film 306 prevents the wetting liquid 308 from spilling out of the cup 304, prior to insertion of the instrument 320 into the biopsy cap system 300. Once the instrument 320 is inserted into the biopsy cap system 300 and pierces the top film 306, the top film 306 includes an opening 322 to allow an instrument 320 to pass through the top film 306, into the opening 324 of the bottom film 310, into the opening 326 of the biopsy cap 302, and through the access port 318. In some embodiments, the top film 306 may be made out of a material and/or have a structure that allows the instrument 320 to pass through the opening 322, yet the opening 322 seals against the outside of the instrument 320 to inhibit leakage of the wetting liquid outside of the biopsy cap 302.

The wetting liquid 308 is placed between the top film 306 and the bottom film 310 of the cup 304. For example, the wetting liquid 308 may include a substance like water, thick water (e.g., a substance intended for dysphagia patient's hydration), a hydrogel-like product, and the like. The wetting liquid 308 wets an outer surface of the instrument 320 as it passes through the opening 322 of the top film 306, then, in order, through the wetting liquid 308, the opening 324 of bottom film 310, the opening 326 of biopsy cap 302, and into the access port 318. The instrument 320 may be removed from the biopsy cap system 300, and another instrument 320 may be inserted in the same biopsy cap system 300. Alternatively, the cup 304 may be removed from the biopsy cap 302, and another cup 304 may be coupled to the biopsy cap 302 prior to insertion of the next instrument 320.

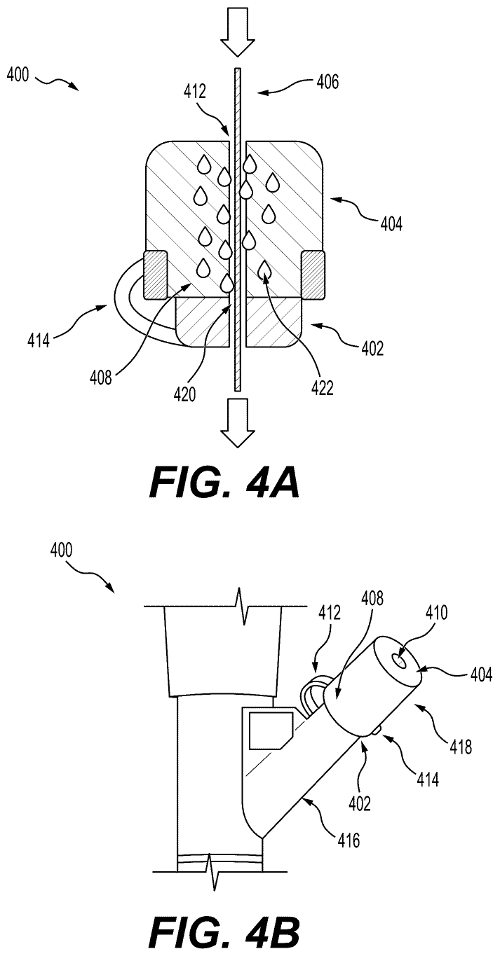

Referring to FIGS. 4A-4B, in one embodiment, a biopsy cap system 400 of this disclosure includes a biopsy cap 402 comprising the same (or similar) elements as outlined in FIGS. 2A-2C. The biopsy cap system 400 also includes a sponge 404 configured to wet an instrument 406 passing through the biopsy cap system 400. FIG. 4A is a cross-sectional view of the biopsy cap system 400 at a plane through a middle of the biopsy cap system 400.

The sponge 404 includes a flange 408, a through hole 410, and a coating 418. The sponge 404 has a substantially cylindrical shape (although other shapes are within the scope of this disclosure) and is made out of a material that is configured to hold a wetting liquid 422. The material may be flexible, compressible, porous, hydrophilic, sterile, and/or disposable. The material may be an open-cell foam. Suitable materials may include polyurethanes, polymers with ester and/or ether functional groups, and composite materials.

The sponge 404 is configured to be placed over the top of the biopsy cap 402. For example, the sponge 404 may include a bottom opening configured to receive the lid 412 of the biopsy cap 402 via the flange 408. The flange 408 is a bottom portion of the sponge 404 that, when fitted over the biopsy cap 402, is located proximal to the biopsy cap 402. The flange 408 includes a first opening to fit around and accommodate the grip 414 and a second opening to fit around and accommodate the biopsy cap lid connector 412. The flange 408 has a dimension and a flexibility to friction fit to the lid 412 of the biopsy cap 402. The flange 408 may flex slightly radially outward upon its coupling to the lid 412 of the biopsy cap 402 to provide the friction fit. The sponge 404 may be removable from the lid 412 of the biopsy cap 402.

The through hole 410 is centrally located within the sponge 404, and has a cylindrical shape with a diameter smaller than the diameter of the sponge 404. The cylindrical shape extends from the top of the sponge 404 to the bottom of the sponge 404 to allow an instrument 406 (e.g., a catheter, a guidewire, etc.) to pass through the through hole 410 of the sponge 404. For example, once the instrument 406 is inserted into the through hole 410, the through hole 410 allows the instrument 406 to pass into the opening 420 of the biopsy cap 402 and through the access port 416. The through hole 410 may have a diameter of approximately 0.05 inches, but the sponge 404 may be flexible enough so that the through hole 410 can accommodate instruments having diameters larger than 0.05 inches.

In some embodiments, the wetting liquid 422 is absorbed by the sponge 404 prior to the sponge's 404 placement on the biopsy cap 402. Alternatively, the wetting liquid 422 is placed on the sponge 404 after the sponge 404 is placed on the biopsy cap 402. The wetting liquid 422 includes a substance like water, thick water (e.g., a substance intended for dysphagia patient's hydration), a hydrogel-like product, and the like. The wetting liquid 422 wets the instrument 406 as it passes through the top opening of the through hole 410, then, in order, through the through hole to contact the wetting liquid 422 in the sponge 404, the bottom opening of the through hole 410, the opening of the biopsy cap 420, and into the access port 416. The instrument 406 may be removed from the biopsy cap system 400, and another instrument 406 may be inserted in the same biopsy cap system 400. Alternatively, the sponge 404 may be removed from the biopsy cap 402, and another sponge 404 may be coupled to the biopsy cap 402 prior to insertion of the next instrument 406.

The coating 418 is affixed to the exterior sides (the outer perimeter) of the sponge 404. In some embodiments, the coating 418 is not affixed to the ends (top and bottom) of the sponge 404, where the through hole 410 begins or ends. In other embodiments, the coating 418 is affixed to those ends, so that the sponge 404 has a coating over all exterior surfaces of the sponge 404. The coating 418 may be made out of a material and/or have a structure that inhibits leakage of the wetting liquid 422 outside of the sponge 404. Suitable coating materials include, for example, plastic, laminated paper, cauterized foam, and silica-based gels.

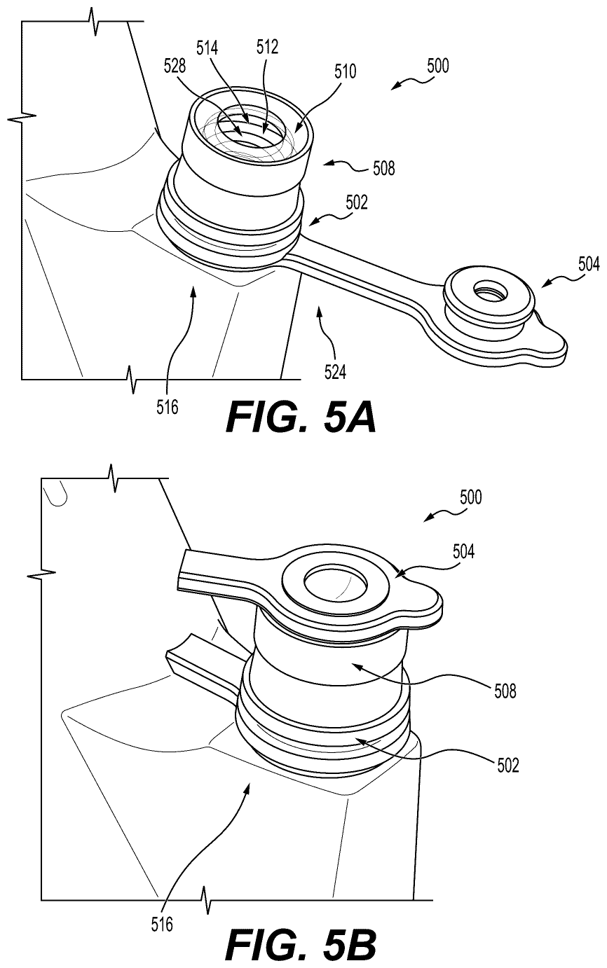

Referring to FIGS. 5A-5B, in one embodiment, a biopsy cap system 500 of this disclosure includes a biopsy cap 524 comprising the same (or similar) elements as outlined in FIGS. 2A-2C. The biopsy cap system 500 also includes a wetting tube 508 configured to wet an instrument passing through the biopsy cap system 500.

The wetting tube 508 includes a substantially cylindrical shape defining an interior to receive and hold a wetting liquid. The wetting tube 508 is configured to be placed over the top 514 of the biopsy cap base 502 and couple to the biopsy cap base 502 via locking geometry. For example, the wetting tube 508 includes a connection groove 510 and a flange 512, much like the groove 219 and the flange 221 shown in FIG. 2C. The wetting tube 508 may include a bottom opening configured to receive the top 514 of the biopsy cap base 502 via the flange 512. The flange 512 may flex slightly radially inward as it inserts into the biopsy cap base 502, and then return to its unflexed position to then fit under a lip of the biopsy cap base 502, much like embodiments describe above. The flange 512 is a bottom portion of the wetting tube 508 that defines the bottom opening within the flange 512. When fitted over the biopsy cap base 502, the flange 512 is located within the biopsy cap base 502.

Additionally, the wetting tube 508 may include a top opening configured to receive the biopsy cap lid 504 via locking geometry, as illustrated in FIG. 5B. The top of the wetting tube 508 may include geometry like the top the biopsy cap base 204/502 (as described above), so that the wetting tube 508 can receive and couple to the biopsy cap lid 504.

A through hole through the wetting tube 508 has a cylindrical shape with a diameter smaller than the diameter of the wetting tube 508. The through hole extends from the top of the wetting tube 508 to the bottom of the wetting tube 508 to allow an instrument (e.g., a catheter, a guidewire, etc.) to pass through the wetting tube 508. For example, once the instrument is inserted into the through hole, the through hole allows the instrument to pass into the top 514 of the biopsy cap base 502 and through the access port 516.

The wetting tube 508 may be removable from the biopsy cap base 502. In other embodiments, the wetting tube 508 may be integrally molded with or otherwise manufactured as one piece with the biopsy cap base 502, where the wetting tube 508 and the biopsy cap base 502 are one structure.

A wetting liquid is placed into the wetting tube 508. For example, the wetting liquid may include a substance like water, thick water (e.g., a substance intended for dysphagia patient's hydration), a hydrogel-like product, and the like. As illustrated in FIG. 5A, the wetting tube 508 may wet the instrument when the top opening has not received the biopsy cap lid 504. For example, the wetting liquid wets an instrument as it passes through the wetting tube 508, then, in order, the biopsy cap base 502 and the access port 516. Alternatively, as illustrated in FIG. 5B, the wetting tube 508 may wet the instrument after the top opening receives the biopsy cap lid 504 via the locking geometry. For example, the wetting liquid wets an instrument as it passes through the biopsy cap lid 504, then, in order, wetting tube 508, the biopsy cap base 502, and the access port 516. The instrument may be removed from the biopsy cap system 500, and another instrument may be inserted in the same biopsy cap system 500. Alternatively, the wetting tube 508 may be removed from the biopsy cap base 502, and another wetting tube 508 may be coupled to the biopsy cap base 502 prior to insertion of the next instrument.

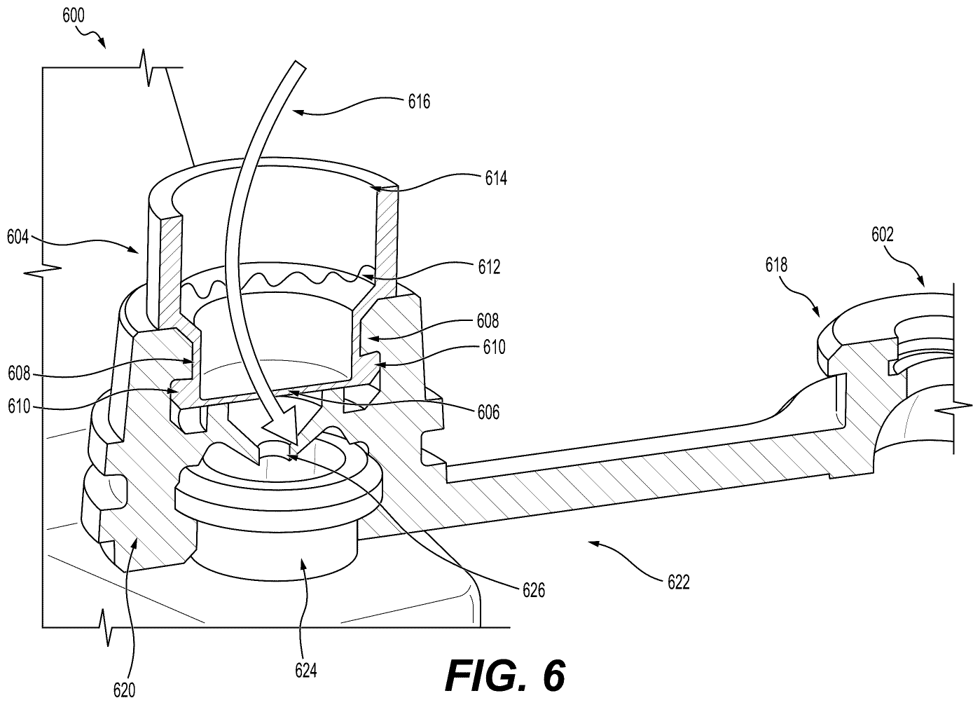

Referring to FIG. 6, in one embodiment, a biopsy cap system 600 of this disclosure includes a biopsy cap 622 comprising the same (or similar) elements as outlined in FIGS. 2A-2C. The biopsy cap system 600 also includes a cup 604 configured to wet an instrument (shown by arrow 616) passing through the biopsy cap system 600.

The cup 604 includes a slit 606, a connection groove 608, a flange 610, a wetting liquid 612, and a main opening 614. The cup 604 includes a substantially cylindrical shape defining an interior to receive and hold the wetting liquid 612. The cup 604 is configured to be placed over the biopsy cap base 620. For example, the cup 604 may include a bottom opening configured to fit inside an opening of the biopsy cap base 620 via the connection groove 608 and the flange 610. The connection groove 608 is a middle portion of the cup 604 that connects a top portion of the cup 604 and the flange 610. The connection groove 608 and the flange 610 have dimensions and a flexibility to friction fit to the opening of the biopsy cap base 620. The flange 610 may flex slightly radially inward as it inserts into the biopsy cap base 620, and then return to its unflexed position to then fit under a lip of the biopsy cap base 620, much like embodiments describe above. The flange 610 is a bottom portion of the cup 604 that defines the bottom opening within the flange 610. When fitted over the biopsy cap base 620, the flange 610 is located within the biopsy cap base 620. The cup 604 may be removable from the biopsy cap base 620. In other embodiments, the cup 604 may be integrally molded with or otherwise manufactured as one piece with the biopsy cap 622, where the cup 604 and the biopsy cap 622 are one structure.

In some embodiments, the cup 604 includes a top film. The top film may be made out of a thin plastic and/or a silicone material so that the instrument 616 (e.g., a catheter, a guidewire, etc.) may pierce through the top film, but firm enough to hold the wetting liquid 612 prior to insertion of the instrument 616. The top film is attached to the top of the cup 604. The top film prevents the wetting liquid 612 from spilling out of the cup 604, prior to insertion of the instrument 616 into the biopsy cap system 600. Once the instrument 616 is inserted into the biopsy cap system 600 and pierces the top film, the top film includes an opening to allow an instrument 616 to pass through the top film, into the slit 606 of the cup 604 and the biopsy cap opening 626, and through the access port 624. In some embodiments, the top film may be made out of a material and/or have a structure that allows the instrument 616 to pass through, yet the top film may have an opening that seals against the outside of the instrument 616 to inhibit leakage of the wetting liquid outside of the cup 604.

The wetting liquid 612 is placed into the cup 604. For example, the wetting liquid 612 may include a substance like water, thick water (e.g., a substance intended for dysphagia patient's hydration), a hydrogel-like product, and the like. The wetting liquid 612 wets an instrument 616 as it passes through the main opening 614, then, in order, through the wetting liquid 612, the slit 606, the biopsy cap base 620, and into the access port 624. The instrument 616 may be removed from the biopsy cap system 600, and another instrument may be inserted in the same biopsy cap system 600. Alternatively, the cup 604 may be removed from the biopsy cap base 620, and another cup 604 may be coupled to the biopsy cap base 620 prior to insertion of the next instrument.

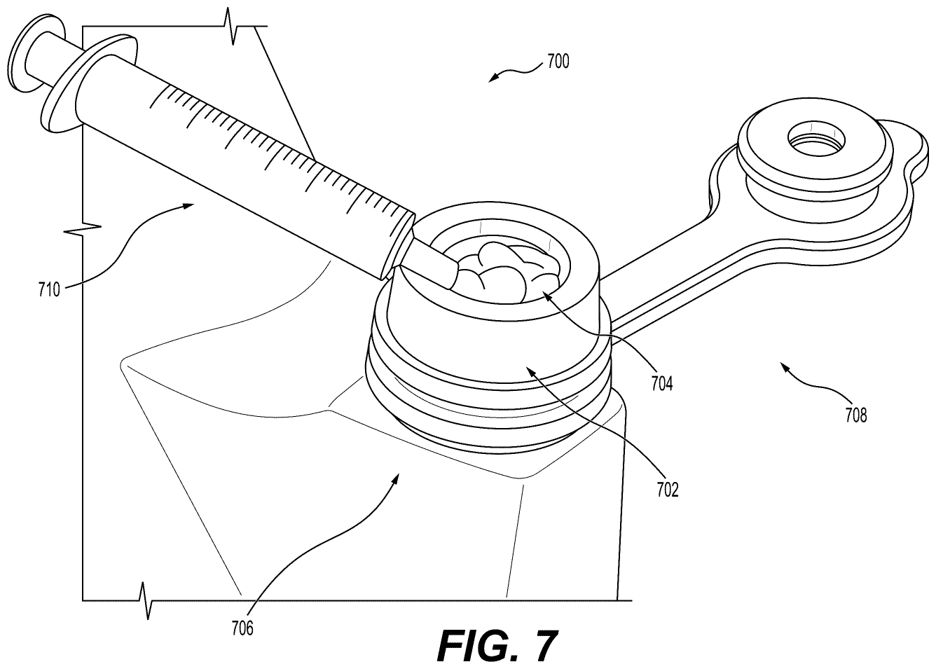

Referring to FIG. 7, in one embodiment, a wetting system 700 of this disclosure includes a biopsy cap 708 comprising the same (or similar) elements as outlined in FIGS. 2A-2C. The wetting system 700 includes a biopsy cap base 702 configured to store a wetting gel 704.

The wetting gel 704 is placed into an opening of the biopsy cap base 702 by an administering device 710 (e.g., a syringe). In some embodiments, the wetting gel 704 is administered through a slit in the biopsy cap 708. The wetting gel 704 may include a hydrogel-like product. The wetting gel 704 wets an instrument as it passes through the wetting gel 704 in the biopsy cap base 702, then through the access port 706. The instrument may be removed from the biopsy cap system 700, and another instrument may be inserted in the same biopsy cap system 700.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented embodiments can be combined to achieve a same or similar result as one or more of the disclosed embodiments. Accordingly, the invention is not to be considered as limited by the foregoing description.

We claim:

1. A cap system, comprising:
a cap for a port of a first medical device; and
a wetting device configured to couple to a portion of the cap and wet a second medical device passing through the cap system.

2. The cap system of claim 1, wherein the wetting device includes a bottom film at a bottom of the wetting device.

3. The cap system of claim 2, wherein the bottom film may be pierced to form an opening in the bottom film.

4. The cap system of claim 1, wherein the wetting device includes a top film at a top of the wetting device.

5. The cap system of claim 4, wherein the top film may be pierced to form an opening in the top film.

6. The cap system of claim 1, further comprising:
a wetting liquid between a top film and a bottom film of the wetting device.

7. The cap system of claim 1, wherein a flange of the wetting device defines a bottom opening configured to receive the portion of the biopsy cap.

8. The cap system of claim 7, wherein the flange is a bottom portion of the wetting device and extends below a bottom film, wherein the bottom opening is radially inward of the flange.

9. The cap system of claim 8, wherein the flange defines a first opening to fit around and accommodate a grip of the cap.

10. The cap system of claim 9, wherein the flange defines a second opening to fit around and accommodate a connector connecting a lid of the cap to a base of the cap.

11. The cap of claim 1, wherein the wetting device comprises a sponge configured to hold a wetting liquid.

12. The cap of claim 11, wherein the sponge is configured to be placed over and coupled to the portion of the cap.

13. The cap of claim 12, wherein the sponge defines a through hole centrally located within the sponge and extending from a top of the sponge to a bottom of the sponge.

14. The cap of claim 13, wherein the through hole has a diameter of approximately 0.05 inches.

15. The cap of claim 12, further comprising a coating on at least one exterior side of the sponge.

16. A wetting device, comprising:
a tube having a substantially cylindrical shape defining an interior to receive and hold a liquid and permit a first medical device to pass through, wherein the tube is configured to be placed over a top of a base of a cap of a port of a second medical device, the tube including bottom locking structure to couple the tube to the base.

17. The wetting device of claim 16, wherein the tube further comprises:
a top opening configured to receive a lid of the cap; and
top locking geometry to couple the tube to the lid.

18. A wetting device, comprising:
a sponge defining a through hole extending within the sponge from a top of the sponge to a bottom of the sponge, the though hole being open at the top of the sponge and open at the bottom of the sponge to allow a first medical device to pass through the through hole, wherein the sponge includes a flange at the bottom of the sponge, the flange defining a bottom opening configured to receive a portion of a cap of a port of a second medical device; and
a coating on an exterior side of the sponge to inhibit leakage of a liquid absorbed by the sponge.

19. The wetting device of claim 18, wherein the flange includes a first opening configured to fit around and accommodate a grip of the cap and a second opening configured to fit around and accommodate a connector connecting a lid of the cap to a base of the cap.

20. The wetting device of claim 18, wherein the wetting device has a substantially cylindrical shape.
